(19) United States
(12) Patent Application Publication
(10) Pub. No.: US 2022/0226511 A1
(43) Pub. Date: Jul. 21, 2022

(54) pH RESPONSIVE COMPOSITIONS AND USES THEREOF

(71) Applicant: The Board of Regents of The University of Texas System, Austin, TX (US)

(72) Inventors: Jinming GAO, Dallas, TX (US); Baran SUMER, Dallas, TX (US); Tian ZHAO, Dallas, TX (US); Zachary T. BENNETT, Dallas, TX (US)

(73) Assignee: The Board of Regents of The University of Texas System, Austin, TX (US)

(21) Appl. No.: 17/614,638

(22) PCT Filed: May 28, 2020
(86) PCT No.: PCT/US2020/034783

§ 371 Date: Nov. 29, 2021

Related U.S. Application Data
(60) Provisional application No. 62/853,593, filed on May 28, 2019.

(51) Int. Cl. A61K 49/00; A61K 9/107; C08G 81/02

(52) U.S. Cl. CPC A61K 49/0054; C08G 81/025; A61K 9/1075; A61K 49/0034

(57) ABSTRACT

Described herein are pH responsive compounds, micelles, and compositions useful for the detection of primary and metastatic tumor tissues. Compounds described herein are imaging agents useful for the detection of primary and metastatic tumor tissue (including lymph nodes). Real-time fluorescence imaging during surgery aids surgeon in the detection of metastatic lymph nodes or delineate tumor tissue versus normal tissue, with the goal of achieving negative margins and complete tumor resection.

### CROSS-REFERENCE TO RELATED APPLICATIONS

This application claims the benefit of U.S. Provisional Patent Application No. 62/853,593 filed on May 28, 2019, which is incorporated herein by reference in its entirety

### STATEMENT AS TO FEDERALLY SPONSORED RESEARCH

This invention was made with the support of the United States government under R01 EB 013149 and CA 192221 by the National Institutes of Health.

### BACKGROUND

Approximately 1.7 million new cancer cases are expected to be diagnosed and approximately 610,000 Americans are expected to die of cancer in 2019. Effective imaging agents are needed for the detection of primary and metastatic tumor tissue.

Treatment guidelines for solid cancers of all stages prominently include surgical removal of the primary tumor, as well as at risk or involved lymph nodes. Despite the biological and anatomical differences between these tumor types, the post-operative margin status is one of the most important prognostic factors of local tumor control and therefore the chance for recurrent disease or tumor metastasis. Surgical excision of solid tumors is a balance between oncologic efficacy and minimization of the resection of normal tissue, and thus functional morbidity. This also holds true for lymphadenectomy performed for diagnostic and therapeutic purposes, often at the same time as the removal of the primary cancer. The presence or absence of lymph node metastasis is the most important determinant of survival for many solid cancers.

Optical imaging strategies have rapidly been adapted to image tissues intra-operatively based on cellular imaging, native auto fluorescence and Raman scattering. The potential of optical imaging include real-time feedback and availability of camera systems that provide a wide view of the surgical field. One strategy to overcome the complexity encountered due to the diversity in oncogenotypes and histologic phenotypes during surgery is to target metabolic vulnerabilities that are ubiquitous in cancer. Aerobic glycolysis, known as the Warburg effect, in which cancer cells preferentially uptake glucose and convert it to lactic acid, occurs in all solid cancers.

Therefore, there remains a need to establish compositions and methods for the determination of the presence of cancer specially cancer metathesis in the lymphatic system.

### SUMMARY

The block copolymers presented herein exploit this ubiquitous pH difference between cancerous tissue and normal tissue and provides a highly sensitive and specific fluorescence response after being taken up by the cells, thus, allowing the detection of tumor tissue, tumor margin, and metastatic tumors including lymph nodes.

Compounds described herein are imaging agents useful for the detection of primary and metastatic tumor tissue (including lymph nodes). Real-time fluorescence imaging during surgery aids surgeon in the detection of metastatic lymph nodes or delineate tumor tissue versus normal tissue, with the goal of achieving negative margins and complete tumor resection. Clinical benefits from the improved surgical outcomes include such as reduced tumor recurrence and re-operation rates, avoidance of unnecessary surgeries, and informing patient treatment plans.

In certain embodiments, provided herein is a block copolymer of Formula (I), or a pharmaceutically acceptable salt, solvate, or hydrate thereof:

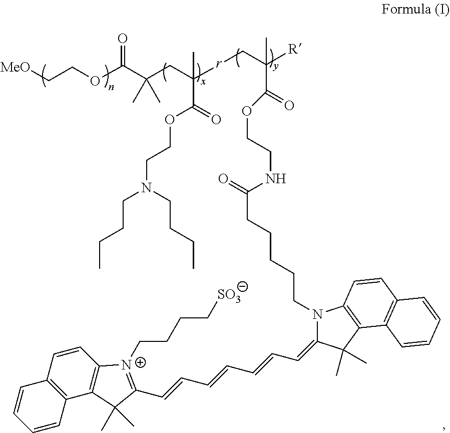

wherein: n is 113; x is 60-150; y is 0.5-1.5, and R′ is a halogen, —OH, or —C(O)OH.

In certain embodiments, provided herein is a micelle comprising one or more block copolymers of Formula (I), or a pharmaceutically acceptable salt, solvate, hydrate, or isotopic variant thereof.

In certain embodiments, provided herein is a pH responsive composition comprising a micelle of a block copolymer of Formula (I), wherein the micelle has a pH transition point and an emission spectra. In some embodiments, the pH transition point is 4-8. In some embodiments, the pH transition point is 6-7.5. In some embodiments, the pH transition point is about 4.8, 4.9, 5.0, 5.1, 5.2, 5.3, 5.4, or 5.5. In some embodiments, a pH transition range (ΔpH_{10-90%}) of less than 1 pH unit. In some embodiments, the emission spectra is between 700-850 nm. In some embodiments, a pH transition range (ΔpH_{10-90%}) of less than 0.25 pH units. In some embodiments, the emission spectra is between 700-850 nm. In some embodiments, a pH transition range (ΔpH_{10-90%}) of less than 0.15 pH units.

In certain embodiments, provided herein is a method of a method of imaging the pH of an intracellular or extracellular environment comprising: (a) contacting a pH responsive composition of the present disclosure with the environment; and (b) detecting one or more optical signals from the environment, wherein the detection of the optical signal indicates that the micelle has reached its pH transition point and disassociated. In some embodiments, the optical signal is a fluorescent signal. In some embodiments, the intracellular environment is imaged, the cell is contacted with the pH responsive composition under conditions suitable to cause uptake of the pH responsive composition. In some embodiments, the intracellular environment is part of a cell. In some embodiments, the extracellular environment is of a tumor or vascular cell. In some embodiments, the extracellular environment is intravascular or extravascular. In some embodiments, the tumor is of a cancer, wherein the cancer the cancer is s breast cancer, head and neck squamous cell carcinoma (NHSCC), lung cancer, ovarian cancer, prostate cancer, bladder cancer, urethral cancer, esophageal cancer, colorectal cancer, brain cancer, or skin cancer. In some embodiments, the tumor is a metastatic tumor cell. In some embodiments, the metastatic tumor cell is located in a lymph node.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1D show the binary fluorescence response of ultra pH sensitive (UPS) polymeric micelle probes. (FIG. 1A) UPS micelles are self-assembled nanoparticles that disassemble into unimers in response to threshold proton concentrations. (FIG. 1B) Structures of amphiphilic block copolymers enable cooperative pH response at specific pKa. (FIG. 1C) Dynamic light scattering shows distinct populations of sizes for unimers (pH below pKa) for USP6.1. (FIG. 1D) Non-linear amplification of fluorescence intensity shows ultra-pH-sensitive response to environmental pH signals. Inset tubes show the near-infrared visualization of UPS5.3-ICG (top), UPS6.1-ICG (middle), and UPS6.9-ICG (bottom) as a function of pH.

FIGS. 2A-2C show in vitro characterization of UPS-ICG nanoparticles. (FIG. 2A) UPS-ICG nanoparticles absorb near-infrared light at λₘₐₓ of 788 nm. (FIG. 2B) Raw mean fluorescence intensity of UPS-ICG nanoparticles measured by LI-COR Pearl 800 nm channel. (FIG. 2C) The number mean diameter of UPS-ICG nanoparticles measured by dynamic light scattering.

FIGS. 3A-3D show whole body near-infrared fluorescence imaging of dissected, tumor-naïve BALB/cj mice enables image-guided resection of LNs in real-time. (FIG. 3A) UPS5.3-ICG and (FIG. 3B) UPS6.1-ICG delineate all the superficial LNs, enabling imaged guided resection. (FIG. 3C) UPS6.9-ICG fluorescence is mostly sequestered to the liver. Image-guided resection of LNs is not permissible. (FIG. 3D) Median fluorescence intensity of LNs is normalized to that of skeletal muscle (Mu). The median CR of anatomical LN group shows dependence on the pKa of polymeric micelle. UPS5.3 shows the highest intensity within each anatomical group of LNs.

FIGS. 4A-4C show pharmacokinetics and organ distribution of UPS nanoparticles in Balb/cj mice. (FIG. 4A) Pharmacokinetics of UPS-ICG fluorescence in collected plasma. Plasma is acidified to show the ‘ON’ state of the nanoparticles. Plasma fluorescence is normalized to fluorescence at time 0 hr, controlling for differences between UPS compositions. (FIG. 4B) Acidified plasma fluorescence is normalized to the collected plasma, showing the ‘ON/OFF Ratio’. (FIG. 4C) Ex vivo imaging of organs after 24 hr circulation of UPS nanoparticles

FIGS. 5A-5C show co-localization of UPS nanoparticles with macrophage sub-populations shows uptake of micelles by lymph node resident macrophages. (FIG. 5A) UPS5.3-ICG co-localizes with CD169 (left), F4/80 (middle), and CD11b (right), but the co-localization is limited within the lymph node. White arrows show co-localization between positive cells and ICG fluorescence. Light gray arrows show staining of F4/80 cells without presence of ICG fluorescence. (FIG. 5B) The pattern of UPS6.1-ICG co-localization with macrophage mirrors that of UPS5.3-ICG. (FIG. 5C) UPS6.9-ICG fluorescence intensity is much lower than UPS5.3-ICG and UPS6.1-ICG. All panels show phagocytosis of nanoparticles by the macrophages in the lymph node but not those in the surrounding tissue. Scale bar is 200 μm.

FIGS. 6A-6F show detection of metastatic lymph nodes with verification by histological examination. (FIG. 6A) A representative 4T1.2-bearing BALB/cj mouse administered with UPS5.3-ICG shows NIRF detection of the primary tumor (P.T.) with whole body imaging as well as delineation of benign (Be), micro-metastatic (Mi), and macro-metastatic (Ma) LNs, enabling image-guided resection of inguinal (In), axillary (Ax), and cervical (Cr) LNs. (FIG. 6B) NIRF imaging of UPS6.1-ICG administered mice shows delineation of the primary tumor and LNs, with the benign LNs appearing nearly as bright as the metastatic LNs. (FIG. 6C) UPS6.9-ICG accumulates at much higher intensity within the liver (Li). Some macro-metastatic LNs are delineated, but many micro-metastatic LNs are undetectable. (FIG. 6D) UPS5.3 signal and median CR of classified tissue shows significance between metastatic and benign LNs. Statistical analysis is done with one-way ANOVA followed by Tukey's multiple comparisons test (*P<0.033, **P<0.0021, ***P<0.0002, ****P<0.0001). (FIG. 6E) UPS6.1 signal and median CR of classified tissue shows significance between macro-metastatic and benign LNs, but the variance in the macro-metastatic distribution is high. (FIG. 6F) UPS6.9 signal and median CR of classified tissue shows significance between macro-metastatic and benign LNs. The signal variable is much lower in intensity compared to UPS5.3 and UPS6.1.

FIGS. 7A & 7B show resection of metastatic lymph nodes in real-time using NIR fluorescence guidance. (FIG. 7A) A 4T1.2-bearing BALB/cj mouse is intravenously injected with UPS5.3-ICG, euthanized, dissected and imaged with the near-infrared camera at 4 fps. All superficial LNs and the primary tumor are delineated. (FIG. 7B) LNs in anatomical regions are visible. A macro-metastatic LN shows increased fluorescence intensity, distinct spatial accumulation of fluorescence, and is larger than other LNs. This LN is resected using the guidance of the NIR fluorescence as feedback. Sampling of other at-risk LNs in the same regional basin is possible. All LN pathology is confirmed by histological examination.

FIGS. 8A-8C show discrimination of metastatic from benign lymph nodes based on ICG patterns. (FIG. 8A) NIRF imaging of benign LNs show ICG fluorescence at the periphery of the nodes. H&E histology and negative pan-cytokeratin stain were used to verify the lack of cancer foci. (FIG. 8B) Micro-metastatic LNs show some UPS5.3-ICG fluorescence in the core of the LN. (FIG. 8C) Macro-metastatic LNs show a broad pattern of ICG fluorescence across the enlarged LN tissue. Pattern of ICG fluorescence correlates with dense cytokeratin staining Upper and lower scale bars are 300 and 50 μm, respectively.

FIGS. 9A-9C show UPS nanoparticle accumulation in macro-metastatic lymph nodes. (FIG. 9A) H&E staining of axillary lymph node shows enlarged nodes. (FIG. 9B) Anti-cytokeratin immunohistochemistry staining reveals presence of cancer foci in the LNs. (FIG. 9C) Near infrared fluorescence scanning of tissue sections reveals UPS5.3-ICG and UPS6.1-ICG accumulate in areas with pan-cytokeratin expression. UPS6.9-ICG displays a much lower fluorescence intensity at the same fluorescent scale as UPS5.3 and UPS6.1. Low scale display show UPS6.9 accumulation in pan-cytokeratin positive regions. Scale bar is 300 μm.

FIGS. 10A & 10B display the receiver operating characteristic (ROC) analysis of metastatic lymph node detection by UPS nanoparticles. (FIG. 10A) ROC curves showing sensitivity and specificity of macro-metastatic LN detection using the LICOR signal of the whole node. UPS5.3 has an AUC of 0.96, indicating high discriminatory capabilities. (FIG. 10B) ROC analysis based on the median CR variable. UPS6.9 has higher discriminatory capability, but it has lower ICG signal as shown in FIG. 6C.

### DETAILED DESCRIPTION OF THE INVENTION

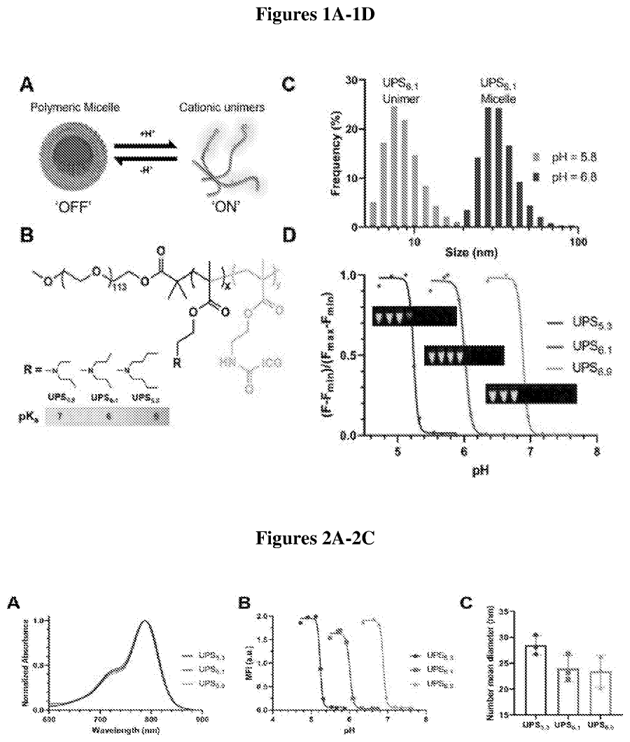

The block copolymers of the invention comprise a hydrophilic polymer segment and a hydrophobic polymer segment, wherein the hydrophobic polymer segment comprises an ionizable amine group to render pH sensitivity. The block copolymers form pH-activatable micellar (pHAM) nanoparticles based on the supramolecular self-assembly of these ionizable block copolymers. At higher pH, the block copolymers assemble into micelles, whereas at lower pH, ionization of the amine group in the hydrophobic polymer segment results in dissociation of the micelle, FIGS. 1A & 1B. Micelle formation and its thermodynamic stability are driven by the delicate balance between the hydrophobic and hydrophilic segments. The ionizable groups may act as tunable hydrophilic/hydrophobic blocks at different pH values, which may directly affect the dynamic self-assembly of micelles. Micellization may sharpen the ionization transition of the amities in the hydrophobic polymer segment, rendering fast and ultra-sensitive pH response.

### I. Block Copolymers

Some embodiments provided herein describe a micelle-based, fluorescent imaging agent. In some embodiments, the micelles comprise a diblock copolymer of polyethylene glycol (PEG) and a dibuthylamino substituted polymethylmethacrylate (PMMA) covalently conjugated to indocyanine green (ICG). In some embodiments, the PEGs comprise the shell or surface of the stable micelle. In some embodiments, the micellar size is <100 nm.

In some embodiments, provided herein is a block copolymer of Formula (I), or a pharmaceutically acceptable salt, solvate, or hydrate thereof:

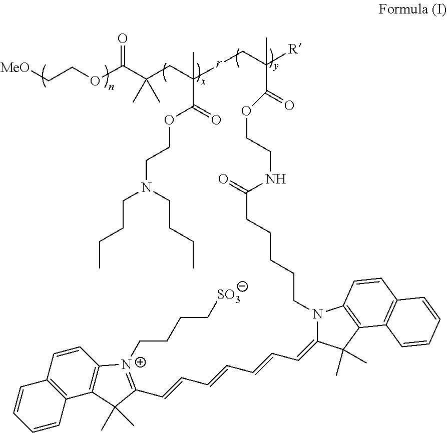

wherein:
n is 113;
x is 60-150;
y is 0.5-1.5; and
R′ is a halogen, —OH, or —C(O)OH.

In some embodiments, the block copolymer of Formula (I) is poly(ethyleneoxide)-b-poly(dibutylaminoethyl methacrylate) copolymer indocyanine green conjugate. In some embodiments, the block copolymer of Formula (I) is PEO113-b-(DBA60-150-r-ICG 0.5-1.5).

Numerous fluorescent dyes are known in the art. In certain aspects of the disclosure, the fluorescent dye is a pH-insensitive fluorescent dyes. In some embodiments, the fluorescent dye is paired with a fluorescent quencher to obtain an increased signal change upon activation. The fluorescent dye, in some instances, is conjugated to the compound directly or through a linker moiety. In some embodiments, the fluorescent dye is conjugated to an amine of the compound through an amide bond. In some embodiments, the fluorescent dye is a coumarin, fluorescein, rhodamine, xanthene, BODIPY®, Alexa Fluor®, or cyanine dye. In some embodiments, the fluorescent dye is indocyanine green, AMCA-x, Marina Blue, PyMPO, Rhodamine Green™, Tetramethylrhodamine, 5-carboxy-X-rhodamine, Bodipy493, Bodipy TMR-x, Bodipy630, Cyanine5, Cyanine5.5, and Cyanine7.5. In some embodiments, the fluorescent dye is indocyanine green (ICG). Indocyanine green (ICG) is often used in medical diagnostics.

In some embodiments, the compound is not conjugated to a dye.

In some embodiments, the block copolymer of Formula (I) is a compound. In some embodiments, the block copolymer of Formula (I) is a diblock copolymer. In some embodiments, is a block copolymer comprises a hydrophilic polymer segment and a hydrophobic polymer segment. In some embodiments, the hydrophilic polymer segment comprises poly(ethylene oxide) (PEO). In some embodiments, the hydrophilic polymer segment is about 2 kD to about 10 kD in size. In some embodiments, the hydrophilic polymer segment is about 3 kD to about 8 kD or about 4 kD to about 6 kD in size. In some embodiments, the hydrophilic polymer segment is about 5 kD in size.

In some embodiments, the hydrophobic polymer segment comprises

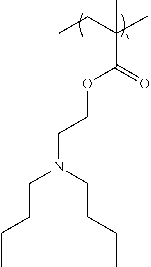

wherein x is about 20 to about 200 in total. In some embodiments, x is about 60-150. In some embodiments, the hydrophilic polymer segment comprises a dibutyl amine.

In some embodiments, R′ is a terminal group. In some embodiments, the terminal capping group is the product of an atom transfer radical polymerization (ATRP) reaction. In some embodiments, R′ is a halogen. In some embodiments, R′ is Br. In some embodiments, R′ is —OH. In some embodiments, R′ is —COH. In some embodiments, R′ is an acid. In some embodiments, R′ is —C(O)OH. In some embodiments, R′ is H.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

### II. Micelles and pH Responsive Compositions

One or more block copolymers described herein may be used to form a pH-responsive micelle and/or nanoparticle. In another aspect, provided herein is a micelle, comprising one or more block copolymers of Formula (I).

The size of the micelles will typically be in the nanometer scale (i.e., between about 1 nm and 1 μm in diameter). In some embodiments, the micelle has a size of about 10 to about 200 nm. In some embodiments, the micelle has a size of about 20 to about 50 nm. In some embodiments, the micelle has a size of less than 100 nm in diameter. In some embodiments, the micelle has a size of less than 50 nm in diameter.

In another aspect, provided herein is a pH responsive composition comprising one or more block copolymers of Formula (I). The pH responsive compositions disclosed herein, comprise one or more pH responsive micelles and/or nanoparticles that comprise block copolymer of Formula (I). Each block copolymer comprises a hydrophilic polymer segment and a hydrophobic polymer segment where the hydrophobic polymer segment comprises an ionizable amine group to render pH sensitivity.

In some embodiments, the pH responsive composition has a pH transition point and an emission spectrum. In some embodiments, the pH transition point is between 4.8-5.5. In some embodiments, the pH transition point is about 4.8, 4.9, 5.0, 5.1, 5.2, 5.3, 5.4, or 5.5. In some embodiments, the pH responsive composition has an emission spectrum between 750-850 nm.

In another aspect is an imaging agent comprising one or more block copolymers of as described here.

### Methods of Use

In some embodiments, the block copolymers and micelles described herein are useful for the detection of primary and metastatic tumor tissues (including lymph nodes), leading to reduced tumor recurrence and re-operation rates.

In some embodiments, the block copolymers and micelles described herein are used in a pH responsive composition or pH responsive micelle. In some embodiments, the pH responsive compositions are used to image physiological and/or pathological processes that involve changes to intracellular or extracellular pH.

Aerobic glycolysis, known as the Warburg effect, in which cancer cells preferentially uptake glucose and convert it into lactic acid, occurs in all solid cancers. Lactic acid preferentially accumulates in the extracellular space due to monocarboxylate transporters. The resulting acidification of the extra-cellular space promotes remodeling of the extracellular matrix for further tumor invasion and metastasis.

Some embodiments provided herein describe compounds that form micelles at physiologic pH (7.35-7.45). In some embodiments, the compounds described herein are conjugated to ICG dyes. In some embodiments, the micelle has a molecular weight of greater than 2×10⁷ Daltons. In some embodiments, the micelle has a molecular weight of ˜2.7×10⁷ Daltons. In some embodiments, the ICG dyes are sequestered within the micelle core at physiologic pH (7.35-7.45) (e.g., during blood circulation) resulting in fluorescence quenching. In some embodiments, when the micelle encounters an acidic environment (e.g., tumor tissues), the micelles dissociate into individual compounds with an average molecular weight of about 3.7×10⁴ Daltons, allowing the activation of fluorescence signals from the ICG dye, causing the acidic environment (e.g. tumor tissue) to specifically fluoresce. In some embodiments, the micelle dissociates at a pH below the pH transition point (e.g. acidic state of tumor microenvironment).

In some embodiments, the fluorescent response is intense due to a sharp phase transition that occurs between the hydrophobicity-driven micellar self-assembly (non-fluorescent OFF state) and the cooperative dissociation of these micelles (fluorescent ON state) at predefined low pH.

In some embodiments, the micelles described herein have a pH transition point and an emission spectra. In some embodiments, the pH transition point is between 4-8. In other embodiments, the pH transition point is between 6-7.5. In other embodiments, the pH transition point is between 4.8-5.5. In certain embodiments, the pH transition point is about 4.8, 4.9, 5.0, 5.1, 5.2, 5.3, 5.4, or 5.5. In some embodiments, the pH transition point is about 5.3. In some embodiments, the pH transition point is about 5.4. In some embodiments, the pH transition point is about 5.5. In some embodiments, the emission spectra is between 400-850 nm. In some embodiments, the emission spectrum is between 700-900 nm. In some embodiments, the emission spectra is between 750-850 nm.

In some instances, the pH-sensitive micelle compositions described herein have a narrow pH transition range. In some embodiments, the micelles described herein have a pH transition range (ΔpH_{10-90%}) of less than 1 pH unit. In various embodiments, the micelles have a pH transition range of less than about 0.9, less than about 0.8, less than about 0.7, less than about 0.6, less than about 0.5, less than about 0.4, less than about 0.3, less than about 0.2, less than about 0.1 pH unit. In some embodiments, the micelles have a pH transition range of less than about 0.5 pH unit. In some embodiments, the pH transition range is less than 0.25 pH units. In some embodiments, the pH transition range is less than 0.15 pH units.

The fluorescence activation ratio is a measure of the ON/OFF state of the micelle. In some embodiments, the fluorescence activation ratio (i.e., the difference between the associated and disassociated micelle) is greater than 75 times of the associated micelle. In some embodiments, the fluorescence signal has a fluorescence activation ratio of greater than 25. In some embodiments, the fluorescence signal has a fluorescence activation ratio of greater than 50.

In some embodiments, the pH responsive micelle has a mean contrast ratio (CR). The mean contrast ratio (CR) is the amount of signal relative to the background signal and is calculated based on Equation 1:

$$Median Contrast Ratio = \frac{Median intensity ( tissue - muscle )}{Standard Deviation ( muscle )} . \qquad ( 1 )$$

In some embodiments, the pH responsive micelle has a high contrast ratio. In some embodiments, the contrast ratio is greater than about 30, 40, 50, 60, 70, 80, or 90. In some embodiments the contrast ratio is great than 50. In some embodiments, the contrast ratio is greater than 60. In some embodiments, the contrast ratio is greater than 70.

In some embodiments, the optical signal is a fluorescent signal.

In some embodiments, when the intracellular environment is imaged, the cell is contacted with the micelle under conditions suitable to cause uptake of the micelle. In some embodiments, the intracellular environment is part of a cell. In some embodiments, the part of the cell is lysosome or an endosome. In some embodiments, the extracellular environment is of a tumor or vascular cell. In some embodiments, the extracellular environment is intravascular or extravascular. In some embodiments, imaging the pH of the tumor environment comprises imaging the sentinel lymph node or nodes. In some embodiments, imaging the pH of the tumor environment allows determination of the tumor size and margins. In some embodiments, the cell may be a cancer cell from a metastatic tumor. In some embodiments, the cancer cell is present in a lymph node. The cancer cell in the lymph node may be used to determine the presence of a metastatic tumor that has spread beyond the original tumor.

In some embodiments the tumor is a solid tumor. In some embodiments, the tumor is of a cancer or carcinoma. Exemplary cancers are selected from but not limited to breast, ovarian, colon, urinary, bladder, lung, prostate, brain, head and neck (NHSCC), colorectal, and esophageal. In some embodiments, the cancer is breast cancer, head and neck squamous cell carcinoma (NHSCC), esophageal cancer, or colorectal cancer. In some embodiments, the cancer is breast cancer, head and neck squamous cell carcinoma (NHSCC), lung cancer, ovarian cancer, prostate cancer, bladder cancer, urethral cancer, esophageal cancer, colorectal cancer, brain cancer, or skin cancer. In some embodiments, the cancer is breast cancer. In some embodiments, the cancer is head and neck squamous cell carcinoma (NHSCC). In some embodiments, the cancer is esophageal cancer. In some embodiments, the cancer is colorectal cancer.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Thrich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the compound of Formula (A) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (A) is prepared as a chloride salt, sulfate salt, bromide salt, mesylate salt, maleate salt, citrate salt or phosphate salt.

In some embodiments, pharmaceutically acceptable salts are obtained by reacting a compound of Formula (A) with a base. In some embodiments, the compound of Formula (A) is acidic and is reacted with a base. In such situations, an acidic proton of the compound of Formula (A) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, melamine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (A), as well as active metabolites of these compounds having the same type of activity.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

As used herein, “pH responsive system,” “pH responsive composition,” “micelle,” “pH-responsive micelle,” “pH-sensitive micelle,” “pH-activatable micelle” and “pH-activatable micellar (pHAM) nanoparticle” are used interchangeably herein to indicate a micelle comprising one or more compounds, which disassociates depending on the pH (e.g., above or below a certain pH). As a non-limiting example, at a certain pH, the compound of Formula (I) is substantially in micellar form. As the pH changes (e.g., decreases), the micelles begin to disassociate, and as the pH further changes (e.g., further decreases), the compound of Formula (I) is present substantially in disassociated (non-micellar) form.

As used herein, “pH transition range” indicates the pH range over which the micelles disassociate.

As used herein, “pH transition value” (pH) indicates the pH at which half of the micelles are disassociated.

A “nanoprobe” is used herein to indicate a pH-sensitive micelle which comprises an imaging labeling moiety. In some embodiments, the labeling moiety is a fluorescent dye. In some embodiments, the fluorescent dye is indocyanine green (ICG).

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. Following longstanding patent law, the words “a” and “an,” when used in conjunction with the word “comprising” in the claims or specification, denotes one or more, unless specifically noted.

### Examples

Compounds are prepared using standard organic chemistry techniques such as those described in, for example, March's Advanced Organic Chemistry, 6^{th} Edition, John Wiley and Sons, Inc. Unless otherwise indicated, conventional methods of mass spectroscopy, NMR, HPLC, protein chemistry, biochemistry, recombinant DNA techniques and pharmacology are employed. Some abbreviations used herein are as follows:

AUC area under the curve

BC breast cancer

CR contrast ratio

HNSCC head and neck squamous cell carcinoma

hr hour(s)

ICG-OSu: indocyanine green succinimide ester

IV intravenous

kg kilogram

LN lymph node

mg milligram(s)

mL milliliters(s)

μg microgram(s)

NC not calculated

NIRF near-infrared fluorescence

ROC receiver operating characteristic

ROI region of interest

SLNB sentinel lymph node biopsy

UPS ultra-pH-sensitive

### Example 1. Materials and Methods

Synthesis of Block copolymer: Block copolymers of Formula (I) described herein are synthesized using standard synthetic techniques or using methods known in the art in combination with methods described in patent publications numbers WO 2012/039741 and WO 2015/188157.

More specifically, ethylpropylaminoethyl methacrylate (EPA), dipropylaminoethyl methacrylate (DPA), and dibutylaminoethyl methacrylate (DBA) were used to synthesize UPS6.9 (PEPA-ICG), UPS6.1 (PDPA-ICG) and UPS5.3 (PDBA-ICG) copolymers by atom transfer radical polymerization (ATRP) from a polyethylene glycol (PEG)-bromide macroinitiator, respectively. ICG-sulfo-OSu (AAT Bioquest) was conjugated to primary amines at a molar ratio of three fluorophores per polymer in methanol for 24 h. Purification with discontinuous diafiltration in methanol using a 10 kDa regenerated cellulose ultrafiltration disc (Amicon Bioseparations) removes unconjugated ICG. ICG-conjugation is quantified by UV-Vis spectroscopy with the Shimadzu UV-1800 at polymer concentration of 10 μg/mL in methanol.

Purified ICG-copolymers in methanol are dispersed in deionized water ten-fold under sonication for micelle self-assembly. Micelles are purified in a 100 kDa centrifugal filter unit (Amicon Bioseparations) with three washes of deionized water. A stock concentration of micelles is maintained at 5.0 mg/mL. Micelle nanoparticles were characterized by dynamic light scattering (DLS) using the Malvern Zetasizer Nano ZS. Micelles were diluted to 0.1 mg/mL in phosphate buffered saline (PBS) at discrete pH (±0.5 pH unit from the polymer pKa, FIG. 1D). Additionally, ICG-fluorescence intensity was measured as a function of pH. Samples were imaged with the LI-COR Pearl in the 800 nm channel at 85 μm resolution.

Animal studies: An orthotopic 4T1.2 BALB/cj model was utilized in eight week old mice. Implantation of 1×10⁶ cells in the fourth, right mammary fat pad resulted in consistent, spontaneous LN metastasis to ipsilateral axillary LNs as well as occasional metastasis to ipsilateral or contralateral cervical and inguinal LNs after 4-5 weeks of primary tumor growth. UPS nanoparticles were administered to 4T1.2-bearing BALB/cj mice intravenously in 0.9% saline at 1.0 mg/kg.

Fluorescence imaging: Real-time fluorescence imaging was performed using an NIRF camera. Emission light was filtered with a 860±12 nm band-pass filter (ThorLabs) and focused with a 25 mm/F1.8 fixed focal length lens (Edmund Optics). Filtered emission wavelengths are detected with the Blackfly S USB3 camera (FLIR) Images were recorded at 4 fps unless otherwise specified. Individual LNs were resected under the guidance of fluorescence imaging system as well as a stereotactic microscope.

Quantitative NIRF imaging was performed with the LI-COR Pearl Small Animal Imaging System. Image acquisition occurs at 85 μm resolution in the 800 nm channel. Quantification occurs in the Image Studio software, drawing ROI with the freehand tool. The median pixel intensity as well as LI-COR signal was exported for each ROI. Fluorescent slides were scanned with the LI-COR Odyssey imager at 21 μm resolution. Images are linked with the same filter for ease of comparison.

Histology: After dissection, LN tissues were formalin-fixed, paraffin-embedded and sectioned in three 5.0 μm slices every 500 μm until tissue exhaustion. This led to three to four groups of three adjacent slides. The first slide is stained with hematoxylin and eosin using an automatic staining instrument (Dakewe). The second slide was used for NIRF imaging. The third adjacent slide was used for pan-cytokeratin immunohisto-chemistry. Heat-induced antigen retrieval was accomplished in Tris pH 9 for 17 min at 110 psi. Slides were blocked for 1 hr with Mouse serum (Mouse on mouse blocking reagent, Vector Laboratories). Anti-mouse pan-cytokeratin antibody (diluted 1:10; AE1/AE3 clone; ThermoFisher) in 2.5% normal horse serum (Vector Laboratories) incubation occurred for 30 min at room temperature. Detection of primary antibody was done for 10 min at room temperature with the Immpress Horse Anti-Mouse IgG Polymer Reagent (Mouse on mouse blocking reagent, Vector Laboratories). The DAB substrate was added until color developed. Benign LNs are classified as pan-cytokeratin negative. Micro-metastases are defined as pan-cytokeratin positive clusters less than 2 mm in size. Macro-metastatic LNs are those with pan-cytokeratin positive clusters greater than 2 mm in size.

Immunohistochemistry staining enables visualization of spatial co-localization between nanoparticles and LN macrophages. BALB/cj mice (8 weeks old) were intravenously injected with 1.0 mg/kg nanoparticle solution in 0.9% saline. LNs were resected under guidance of the an NIRF camera system. LNs were embedded in OTC medium and frozen with liquid nitrogen. Frozen sections were sectioned at 12 μm at intervals of 500 μm. Sections were fixed in −20° C. acetone for 10 min followed by 10 min of drying at room temperature. Next, sections were washed twice in 1×PBS for 5 min each. Blocking occurred with normal goat serum for 1 hr. Aspiration of the blocking serum was followed by incubation of primary antibodies: FITC anti-mouse CD169 (1:125; Clone 3D6.112; Lot no. B271952), PE anti-mouse F4/80 (1:50; Clone BM8; Lot no. B199614), and APC anti-mouse CD11b (1:50; Clone M1/70; Lot no. B279418). All antibodies were multiplexed in PBS with 0.5% Tween and added to each tissue section. Incubation occurs overnight at 4° C. Sections were washed three times in PBS for 5 min each. Mounting cover slips were used with Diamond Mount with DAPI. Slides were imaged with the Keyence Automated Microscope.

Statistical Analysis: LI-COR signal and median CR values were grouped according to histological status. Each group (benign, micro-metastatic, and macro-metastatic) was analyzed with a one-way ANOVA for statistical difference of means. A Tukey multiple comparison assessed differences between the mean of each group. An ‘ROC Curve’ module with the ‘Wilson/Brown’ method was used in GraphPad Prism to compare discrimination between variables and groups. This statistic was maximized to determine the threshold for sensitivity and specificity.

### Example 2. pH Sensitive Nanoparticles Show Cooperative Fluorescence Response to Environmental pH

Three ultra pH sensitive (UPS) block copolymers were synthesized. Copolymers with discrete pH-transitions to cover a range of pH response (UPS5.3, UPS6.1, and UPS6.9; each subscript indicates the apparent pKₐ value) (FIG. 1B, Table 1). In particular, the amphiphilic block copolymer UPS6.1 has a pKₐ at 6.1. At pH-values above the pKₐ, UPS6.1 self-assembles into 24.0±2.1 nm micelles (FIG. 1C, Table 1). Below pH-values of 6.1, protonation of polymer chains causes micelle disassembly into 4.9±1.2 nm unimers (FIG. 1C). UPS5.3 (28.5±1.5 nm) and UPS6.9 (23.4±2.5 nm) also have sharp pH-dependent micelle-to-unimer transitions as well (Table 1, FIG. 2C). The comparable nanoparticle size (23-28 nm) and identical PEG length (5 kDa) between micelle compositions are important to keep size and surface chemistry consistent in LN targeting, enabling the specific evaluation of pH-thresholds in the detection of LN metastases.

TABLE 1
Characterization of PEG-b-(PR-r-Dye) nanoprobes.

| PR-Dye | Particle Size (nm)^{a} | pHt^{b} | ΔpH10-90%^{c} |
|---|---|---|---|
| UPS5.3-ICG (PDBA) | 28.5 ± 1.5 | 5.3 | 0.28 |
| UPS6.1-ICG (PDPA) | 24.0 ± 2.1 | 6.1 | 0.33 |
| UPS6.9-ICG (PEPA) | 23.4 ± 2.5 | 6.9 | 0.24 |

^{a}Number-based size determined by dynamic light scattering.
^{b}Determined by ICG fluorescence using the LI-COR Pearl Imager.
^{c}Determined by NaOH-titration.

To report local pH values, each polymer was conjugated with indocyanine green (ICG), a fluorophore that is approved by the FDA and compatible with clinical, near infrared (NIRF) imaging systems. Each UPS-ICG nanoparticle shows comparable copies of dye per polymer (Table 1, FIG. 2A). However, in the micelle state at pH 7.4, homoFRET-induced quenching abolishes the ICG fluorescence signal. At pH below the pKₐ, UPS micelles disassemble into individual unimers and amplify fluorescence intensity over 50-fold within a 0.3 pH span (FIG. 1D, Table 2). The USP nanoparticle display binary encoding of pH-thresholds by NIRF (FIGS. 1D, 2A, and 2B, Table 2). This ‘digital’ signal represents fluorescence activation as a discrete value (ON=1, OFF=0) at different pH-threshold.

TABLE 2
Measurement of conjugation efficiency and quantum
yields of dye-conjugated copolymers.

| | Dye Conjugation | | |
|---|---|---|---|
| | Dye per | Efficiency | ON/OFF |
| PR-Dye | polymer (x)^{a} | (%)^{a} | Ratio^{b} |
| P(DBA₇₀-r-ICGₓ) | 1.9 | 0.63 | 56 |
| P(DPA₆₅-r-ICGₓ) | 2.0 | 0.62 | 59 |
| P(EPA₁₁₅-r-ICGₓ) | 1.8 | 0.76 | 39 |

^{a}Determined by a standard curve base on UV-Vis spectroscopy of the free ICG in methanol.
^{b}Determined by the ICG fluorescence emission in 1 × PBS using the LI-COR Pearl Imager.

### Example 3. Real-Time Systemic Lymphatic Mapping in Tumor Naïve Mice Guides Resection of LNs

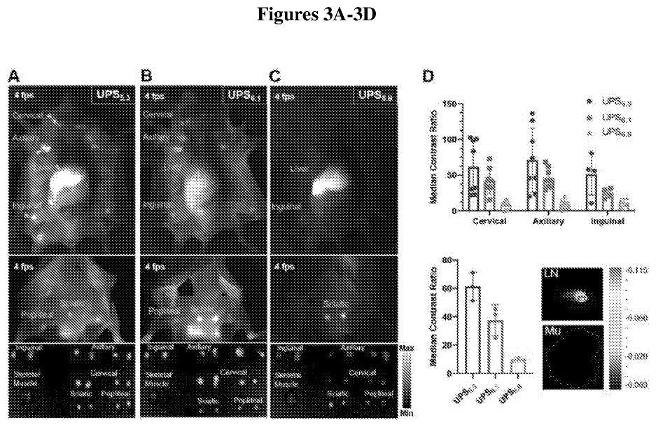

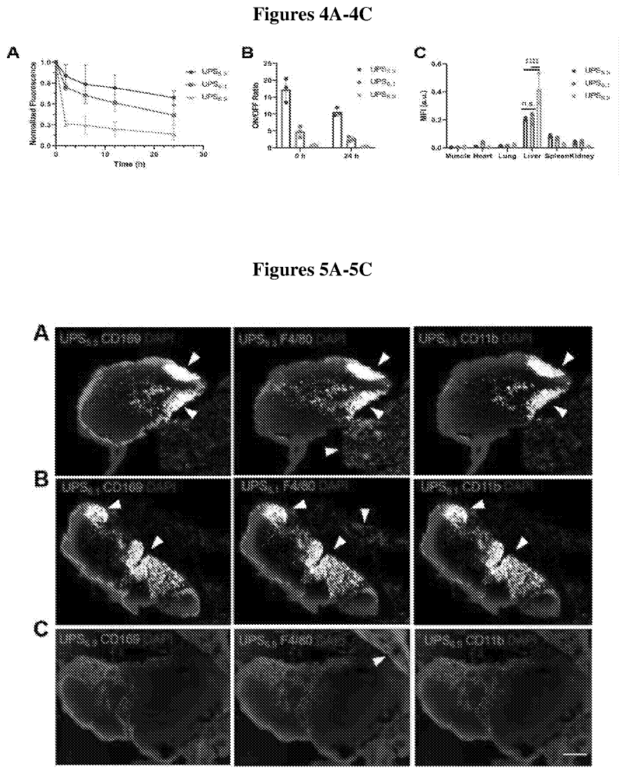

Each polymeric nanoparticle formulation was intravenously administered in tumor-naïve BALB/cj mice to evaluate whole-body lymphatic mapping. NIRF imaging visualizes dissected mice, clearly delineating LNs in the UPS5.3 and UPS6.1 administered animals (FIGS. 3A & 3B). This delineation facilitates image-guided resection of all superficial LNs in real-time. Quantitative imaging of resected tissue ex vivo with the LI-COR Pearl shows comparable ICG signals from different anatomical groups of LNs. The median contrast ratio (CR) was calculated for all LN tissues (Equation 1):

$$Median Contrast Ratio = \frac{Median intensity ( tissue - muscle )}{Standard Deviation ( muscle )} . \qquad ( 1 )$$

LN fluorescence was amplified with a pan-LN median CR of 63.3 for UPS5.3 and 39.9 for UPS6.1 (FIG. 3D). The UPS6.9 median CR value was significantly lower at a value of 10.7 (FIG. 3D).

To explain the differences between micelle compositions in LN targeting, a pharmacokinetics study was performed evaluating fluorescence in tumor-naïve BALB/cj blood plasma after intravenous injection (FIG. 4A). UPS6.9 was quickly cleared from the blood compared to UPS5.3 and USP6.1 (FIG. 4A). In addition, UPS6.9-ICG has low ON/OFF ratio after acidification of blood plasma, indicating UPS6.9 disassembles 24 hr after intravenous injection (FIG. 4B). All nanoparticles are stable over 24 hr with high ON/OFF ratios during incubation in normal mouse serum. The low ON/OFF ratio of UPS6.9 is attributed to the fast clearance of the nanoprobes in the liver (FIG. 4C), which results in lower serum concentration and increased thermodynamic propensity to disassemble.

Biodistribution of micelles to LNs appears to be a critical parameter for discrimination of metastatic LNs. UPS6.9 has a lower blood half-life than UPS6.1 and UPS5.3 as shown by increased accumulation in the liver in both tumor-bearing and tumor-naïve mice. To investigate further the effect of biodistribution and circulation time on LN metastasis detection, additional circulation times of 6 hr and 72 hr after intravenous administration of UPS5.3 nanoparticles were included. Sinusoidal macrophage takes up nanoparticles quickly as the ‘halo’ phenomenon is present in LNs from the 6 hr group. However, it does not appear longer circulation time permits increased discrimination of LN metastasis. Overall, the increased half-life of UPS5.3 enables comparatively better ‘capture and integration’ of ICG fluorescence within the lymph node metastasis microenvironment.

### Example 4. LN-Resident Macrophages Internalize UPS Polymeric Micelles

While NIRF imaging delineates all superficial LNs, the lymphotropic delivery mechanism is unclear. Because phagocyte-containing reticuloendo-thelial systems (e.g., liver, spleen) have increased fluorescence intensity, it is theorized that LN-resident macrophages are responsible for the uptake of UPS micelles, leading to amplification of ICG fluorescence signals. Multiplexed immunohistochemistry (IHC) staining of distinct macrophage populations was utilized along with visualization of UPS nanoparticle uptake. UPS5.3-ICG and UPS6.1-ICG fluorescence signals appear in distinct regions in the LN (FIGS. 5A & 5B). These regions show significant overlap with LN-resident macrophages specifically, CD169⁺/F4/80⁺/CD11b⁺ macrophages co-localize with UPS5.3-ICG fluorescence. These cells share the same biomarkers as LN-resident macrophage. Additionally, ICG fluorescence does not overlap with F4/80′ macrophages in the adjacent tissues surrounding the LN, supporting the assumption of LN-specific delivery (FIGS. 5A & 5B), indicating only LN-resident macrophage sequester UPS nanoparticles.

### Example 5. Detection of Metastatic LNs in Tumor-Hearing Mice

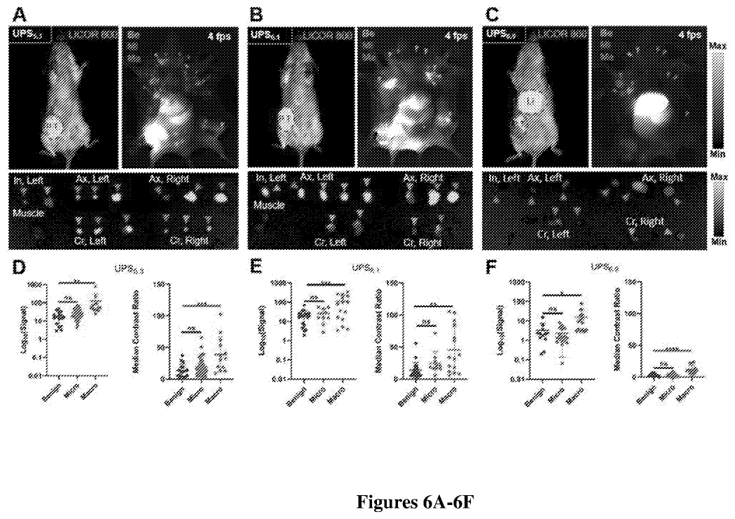

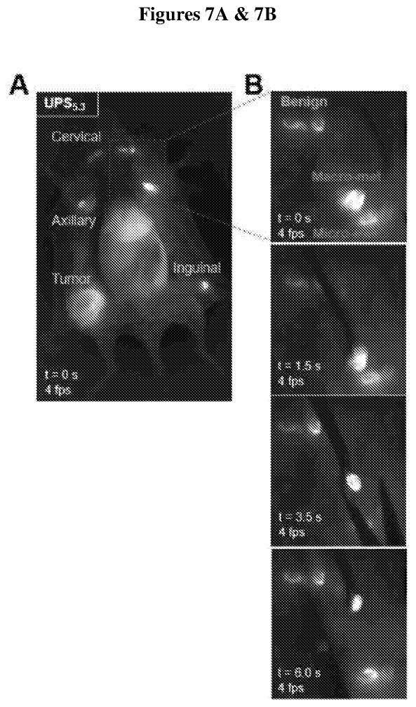

The differences in fluorescence intensity of metastatic LNs against benign LNs was quantified using the syngeneic 4T1.2-BALB/cj murine model. UPS5.3, UPS6.1, or UPS6.9 nanoparticles were intravenously administered at the same dose (1.0 mg/kg) for systemic detection of LN metastases. NIRF imaging of live mice by the LICOR Pearl, after 24 h circulation, showed fluorescence emission within the primary tumor but not metastatic LNs (top left panels, FIGS. 6A-6C). In contrast, NIRF imaging of dissected mice shows accumulation in LNs in addition to primary tumors (top right panels, FIGS. 6A-6C). UPS5.3 and UPS6.1 administered animals show bright fluorescence signal in all superficial LNs (FIGS. 6A & 6B). UPS6.9 administered animals show micelle accumulation in enlarged LNs (FIG. 6C). Real-time fluorescence imaging enabled guided resection of all LNs (FIGS. 7A & 7B). Macro-metastatic LNs are often distinct in fluorescence intensity, spatial pattern, and size from other LNs, enabling precision resection of these LNs (FIG. 7B).

The median contrast ratio was quantified for all resected tissue (Equation 1). Additionally, the LI-COR Signal was used to quantify the total fluorescence intensity from a region of interest (ROI). Each variable conveys distinct information. Median CR evaluates the pixel-based, median fluorescence intensity of LNs whereas LI-COR signal reports the summated fluorescence intensity of the LN tissue. Both variables were evaluated in statistical analysis of grouped tissue. Histological examination of LNs allowed for grouping of tissue based on pathology. LNs were classified as either benign, micro-metastatic (cancer foci <2 mm), or macro-metastatic (cancer foci >2 mm). Median CR and LI-COR signal values were grouped accordingly (FIGS. 5D-F). There is a significant difference between benign and macro-metastatic groups (FIGS. 5D-F). However, no micelle groups display a significant difference between benign and micro-metastases.

### Example 6. UPS Nanoparticles Accumulate within the Cancer Foci of Metastic LNs

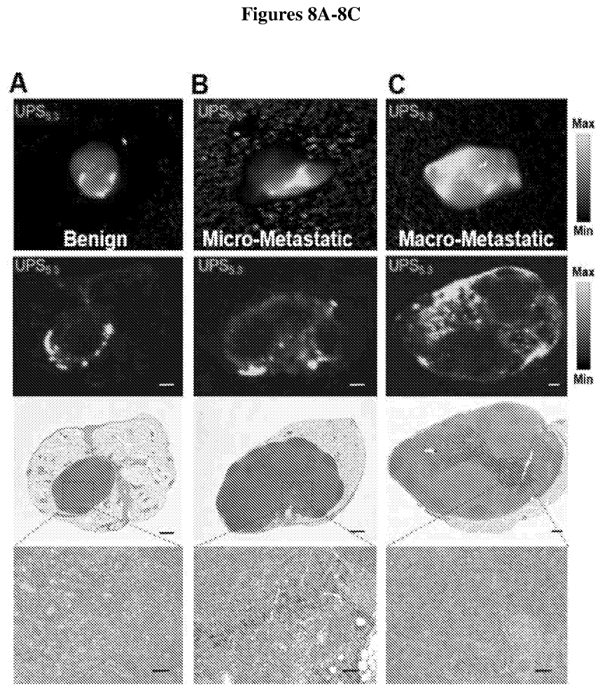

In addition to differences in fluorescence intensity, different patterns of fluorescence signal between benign LNs and macro-metastatic LNs were identified. Benign LNs display a ‘halo’ of UPS5.3-ICG intensity by both real-time imaging ex vivo imaging (FIGS. 7A, 7B, & 8A). Histological analysis confirms no pan-cytokeratin clusters in this LN subset (FIG. 8A). Moreover, microscopic imaging confirms the accumulation of UPS nanoparticles at the edges of LN tissue (FIG. 8A). This pattern is also apparent with UPS6.1 and UPS6.9 administered animals. The peripheral distribution of UPS5.3 nanoparticles in benign LNs colocalizes with LN-resident macrophages in the LN sinusoids. These results are in agreement with the fluorescence localization in tumor-naïve LNs (FIG. 4). However, in benign LNs from tumor-bearing mice, CD1b⁺ macrophages appear more motile within the surrounding tissue compared to the same population in tumor-naïve mice.

Micro-metastatic LNs show a spectrum of fluorescence signatures. Fluorescence may localize to LN edges or show uniform fluorescence across small cancer foci. A mixed pattern with both fluorescence localization at edges and within pan-cytokeratin clusters is the most typical signature (FIG. 8B). In contrast, macro-metastatic LNs display a broad pattern of fluorescence intensity (FIG. 8C). Microscopic analysis shows the ICG signal overlaps mostly with anti-cytokeratin staining (FIG. 8C), indicating cancer-specific accumulation of UPS unimers. Similar result with the UPS6.1 administered group were observed. Moreover, fluorescence intensity of metastatic LN tissue from the UPS6.9 group is decreased compared to UPS6.1 and UPS5.3 (FIG. 9).

All three micelles, display accumulation in pan-cytokeratin positive cancer foci, resulting in detectable fluorescence signals. Quantification of fluorescence intensity reveals LICOR signal is an appropriate metric to achieve discrimination of LN metastasis, especially in the UPS5.3 group. Although LN-resident macrophage uptake of UPS nanoparticles causes background fluorescence, the resulting fluorescence intensity is quantifiably distinct from metastatic LNs. Macrophages internalize micelles upon delivery to LNs and amplify the fluorescence within their acidic organelles. Conversely, metastatic LNs show a broad pattern of fluorescence throughout the LN cortex correspondent with cancer-foci. This pattern of activation could be detectable by the surgeon during resection. There is potential to utilize both intensity and spatial localization of fluorescence to achieve greater discrimination of metastatic LNs.

### Example 7. ROC Discrimination of Metastatic LNs from Benign LNs

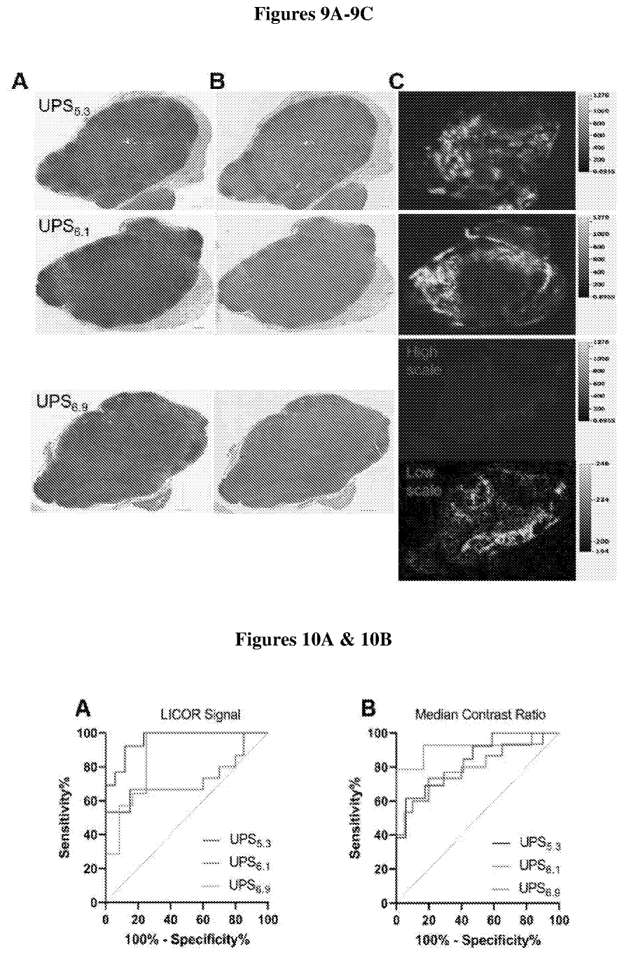

The receiver operating characteristic (ROC) of macro-metastatic LN detection were quantified (Table 3). Quantifying tissue with size-dependent LI-COR signal reveals UPS5.3 has high discriminatory power (AUC=0.96; sensitivity=92.3% and specificity=88.2%) of macro-metastatic LNs over benign LNs (FIG. 10A). Discrimination of benign LNs from macro-metastatic LNs is also feasible using median CR for each polymer (FIG. 10B). The data indicates a lack of discrimination of micro-metastases over benign LNs with either median CR or LICOR signal.

TABLE 3
Receiver operating characteristic analysis of benign
versus micro-metastatic LNs for UPS nanoparticles.

| Micelle | Groups | Variable | Sensitivity (%) | Specificity (%) | AUC |
|---|---|---|---|---|---|
| UPS5.3 | Benign (n = 17) | Signal | 69.2 | 58.8 | 0.67 |
| | Micro-met (n = 39) | Median CR | 87.2 | 58.8 | 0.64 |
| UPS6.1 | Benign (n = 20) | Signal | 50.0 | 75.0 | 0.58 |
| | Micro-met (n = 10) | Median CR | 90.0 | 70.0 | 0.76 |
| UPS6.9 | Benign (n = 12) | Signal | 64.7 | 66.7 | 0.60 |
| | Micro-met (n = 17) | Median CR | 55.6 | 66.7 | 0.55 |

UPS = ultra-pH-sensitive;
CR: contrast ratio;
AUC = area under the curve

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A block copolymer of Formula (I), or a pharmaceutically acceptable salt, solvate, hydrate, or isotopic variant thereof: 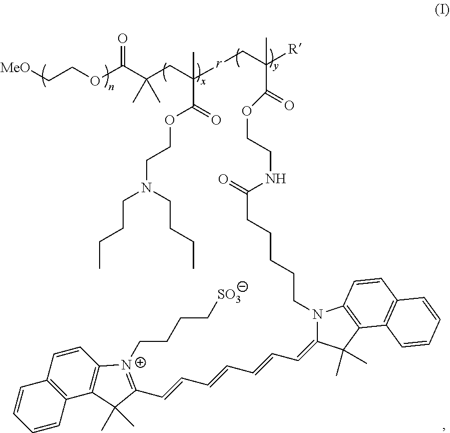
wherein:
n is 113;
x is 60-150;
y is 0.5-1.5; and
R′ is a halogen, —COH, or —C(O)OH.

2. A micelle comprising of one or more block copolymers according to claim 1.

3. A pH responsive composition comprising a micelle of claim 2, wherein the micelle has a pH transition point and an emission spectrum.

4. The pH responsive composition of claim 3, wherein the pH transition point is between 6-7.5.

5. The pH responsive composition of claim 3, wherein the pH transition point is about 4.8, 4.9, 5.0, 5.1, 5.2, 5.3, 5.4, or 5.5.

6. The pH responsive composition according to any one of claims 3-5, wherein the emission spectrum is between 700-850 nm.

7. The pH responsive composition according to any one of claims 3-6, wherein the composition has a pH transition range (ΔpH_{10-90%}) of less than 1 pH unit.

8. The pH responsive composition of claim 7, wherein the pH transition range is less than 0.25 pH units.

9. The pH responsive composition of claim 7, wherein the pH transition range is less than 0.15 pH units.

10. The pH responsive composition according to any one of claims 3-9, wherein the pH responsive composition has a fluorescence activation ratio of greater than 25.

11. The pH responsive composition according to any one of claims 3-10, wherein the pH responsive composition has a fluorescence activation ratio of greater than 50.

12. The pH responsive composition according to any one of claims 3-11, wherein the pH responsive composition has a mean contrast ratio of greater than 50.

13. An imaging agent comprising one or more block copolymers of claim 1.

14. The imaging agent of claim 13 comprising poly(ethyleneoxide)-b-poly(dibutylaminoethyl methacrylate) copolymer indocyanine green conjugate.

15. A block copolymer comprising a hydrophilic polymer segment and a hydrophobic polymer segment, wherein the hydrophilic polymer segment comprises poly(ethylene oxide) (PEO) and the hydrophobic polymer segment comprises 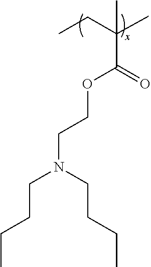
wherein x is about 20 to about 200 in total.

16. The block copolymer of claim 15, wherein x is 60-150.

17. A method of imaging the pH of an intracellular or extracellular environment comprising:
(a) contacting a pH responsive composition of claims 3-12 with the environment; and
(b) detecting one or more optical signals from the environment, wherein the detection of the optical signal indicates that the micelle has reached its pH transition point and disassociated.

18. The method of claim 17, wherein the optical signal is a fluorescent signal.

19. The method of claim 17 or 18, wherein when the intracellular environment is imaged, the cell is contacted with the pH responsive composition under conditions suitable to cause uptake of the pH responsive composition.

20. The method of any one of claims 17-19, wherein the intracellular environment is part of a cell.

21. The method of any one of claims 17-19, wherein the extracellular environment is of a tumor or vascular cell.

22. The method of claim 21, wherein the extracellular environment is intravascular or extravascular.

23. The method of claim 21, wherein the tumor is of a cancer.

24. The method of claim 23, wherein the cancer is the cancer is s breast cancer, head and neck squamous cell carcinoma (NHSCC), lung cancer, ovarian cancer, prostate cancer, bladder cancer, urethral cancer, esophageal cancer, colorectal cancer, brain cancer, or skin cancer.

25. The method of claim 21, wherein the tumor is a metastatic tumor cell.

26. The method of claim 25, wherein the metastatic tumor cell is located in a lymph node.
